SEQUENCE LISTING (20 sequences)

SEQ ID NO: 1 - DNA, 337 bp, Escherichia coli
gtgtggcttttgtgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgagcacgtca

SEQ ID NO: 2 - DNA, 740 bp, Escherichia coli
gacctgttaaaacttcgcgaagtgatagggcagtataaagggtacagtgtgaaattccagactcttacctgattgatgattctctattctcccgtgatatttatcatcctgcattgggggaaatagatgtacaagcgccattttgctgtacaaataacctacaaaaagcccaacaaaaataattgcaaataaacaaagattgctggaaattatgcggatgttaattatttgtgaaatagttaacaagcgttatagtttttctgtggtagcacagaataatgaaaagtgtgtaaagaagggtaaaaaaaaccgaatgcgaggcatccggttgaaataggggtaaacagacattcagaaatgaatgacggtaataaataaagttaatgatgatagcgggagttattctagttgcgagtgaaggttttgttttgacattcagtgctgtcaaatacttaagaataagttattgattttaaccttgaattattattgcttgatgttaggtgcttatttcgccattccgcaataatcttaaaaagttcccttgcatttacattttgaaacatctatagcgataaatgaaacatcttaaaagttttagtatcatattcgtgttggattattctgcatttttggggagaatggacttgccgactgattaatgagggttaatcagtatgcagtggcataaaaaagcaaataaaggcatataacagagggttaataacatgaaagttaaa

SEQ ID NO: 3 - DNA, 457 bp, Escherichia coli
tggcacaggaacgttatccggacgttcagttccaccagacccgcgagcattaattcttgcctccagggcgcggtacgcgctgcgccctgtcaatttcccttccttattagccgcttacggaatgttcttaaaacattcacttttgcttatgttttcgctgatatcccgagcggtttcaaaattgtgatctatatttaacaaagtgatgacatttctgacggcgttaaataccgttcaatgcgtagatatcagtatctaaagccgtcgattgtcattctaccgatattaataactgattcagaggctgtaatggtcgttattcatcactcatcgcttttgtgatggcgaccattgacttctgtagagggtgaagtctctccctattcagcaatgcaacctcgtgttgccaggctcaaattacgagcaaacatacaggaataaatcgatgactatgaca

SEQ ID NO: 4 - DNA, 385 bp, Escherichia coli
cccgcggagttccaccccggggctaccgctcccgatacgctgccaatcagttaacaccaggtcctggagaaaccgcttttgtggtgaccaacatacgagcggctctatagatagtgtaggagatcaggttgttttttttccagaaggttaaccactatcaatatattcatgtcgaaaatttgtttatctaacgagtaagcaaggcggattgacggatcatccgggtcgctataaggtaaggatggtcttaacactgaatctttacggctgggttagccccgcgcacgtagttcgcaggacgcgggtgacgtaacggcacaagaaacgctagctggccagtcatcgacaactttatggaaggagtaacactatggcttataaacac

SEQ ID NO: 5 - DNA, 230 bp, Escherichia coli
tacgtcgcactgtggcggctatcgcactttaacgtttcgtgctgccccctcagtctatgcaatagaccataaactgcaaaaaaaagtccgctgataaggcttgaaaagttcatttccagacccatttttacatcgtagccgatgaggacgcgcctgatgggtgttctggctacctgacctgtccattgtggaaggtcttacattctcgctgatttcaggagctattgatt

SEQ ID NO: 6 - DNA, 280 bp, Escherichia coli
acttttatccactttattgctgtttacggtcctgatgacaggaccgttttccaaccgattaatcataaatatgaaaaataattgttgcatcacccgccaatgcgtggcttaatgcacatcaacggtttgacgtacagaccattaaagcagtgtagtaaggcaagtcccttcaagagttatcgttgatacccctcgtagtgcacattcctttaacgcttcaaaatctgtaaagcacgccatatcgccgaaaggcacacttaattattaaaggtaatacact

SEQ ID NO: 7 - DNA, 259 bp, Escherichia coli
taaaagcgtgttatactctttccctgcaatgggttccgtagcagggaaagagaccccgttgtctcttcccggtatttcatctctatgtcacattttgtgcgtaatttattcacaagcttgcattgaacttgtggataaaatcacggtctgataaaacagtgaatgataacctcgttgctcttaagctctggcacagttgttgctaccactgaagcgccagaagatatcgattgagaggatttgaatgactgacaaaatg

SEQ ID NO: 8 - DNA, 115 bp, Escherichia coli
acttgatactgtatgagcatacagtataattgcttcaacagaacatattgactatccggtattacccggcatgacaggagtaaaaatggctatcgacgaaaacaaacagaaagcg

SEQ ID NO: 9 - DNA, 102 bp, Escherichia coli
cttttcacattaatctggtcaataaccttgaataattgagggatgacctcatttaatctccagtagcaactttgatccgttatgggaggagttatgcgtctg

SEQ ID NO: 10 - DNA, 224 bp, Escherichia coli
ataactcgtaacgccaattcttacttttccgccttcacaaatgccgccactcaaacagagcggcatttttcttccccgcaacattcaattctgttttgcgtgcctgctccagattttgcgatgtttttttgcccagcacactgagaacgtgagatactcacaactgtatataaatacagttacagatttactttctttgcaattgatatcacatggagtgggca

SEQ ID NO: 11 - DNA, 148 bp, Escherichia coli
attggcacttctactccgtaattggcaagacaaacgagtatatcaggcattggatgtgaataaagcgtataggtttacctcaaactgcgcggctgtgttataatttgcgacctttgaatccgggatacagtagagggatagcggttag

SEQ ID NO: 12 - DNA, 80 bp, Escherichia coli
aagattgttggtttttgcgtgatggtgaccgggcagcctaaaggctatccttaaccagggagctgattatgaaaaaagcc

SEQ ID NO: 13 - DNA, 938 bp, Escherichia coli
cttgttggttttgtgtttaacaatatttatacaagcacagctttacaggggagacaatggaaaatttttcagcaagggaaaattgaggggttgatcacgttttgtactgaattgcagataacaaaaaaccccgccggagcgaggtttcgtcagtcgcctgcggctggtaaccgcaaagcacactgtattatgtcaacactgaaagtatacgtgttccgcgcagaacgcgcaatttcggcacgaattttgacgtatttagtgcatagttgagtatcgatcacagtttgcgttttgtccaaatattactgtttatttatacagtaaacttctataatatcactgtacgcaatgtgttatgcgggggccgcatcgttacccggcgcactaagtcctggctgaaacgggtggtgccgtcagcgccttaaccccgcgtgagcacactgtgttatgtcaacaagcacaacgtttctccttgagataccgcgtgcacaacagctggcaacaggcagcggaaaggtacgtcagctggcagtgctcctgaaccacaggagacgcgtatgaacctggtggatatcgccattcttatcctcaaactcattgttgcagcactgcaactgcttgatgctgttctgaaatacctgaagtaattcagattcaagtcgcaccaaaggggagcgggaaaccgctccccttttatatttagcgtgcgggttggtgtcggatgcgatgctgacgcatcttatccgccctaccatctctcccggcaacatttattgccgcttttgtttacatattctgccgctaaacaattccccattcctggcgtatatctggctaacattcatcaatgtgatagattcctctcccgcatttatgggaatgcgtagtgacttattctaattatttttataaaagcatccgtgataatgaaaaggcaa

SEQ ID NO: 14 - DNA, 309 bp, Escherichia coli
ccagcataacttcccgacgcaaagtgattaaaagggagccaatacaggcaagtcgttgagaataaagtgcaggttaaactgggtaaagcggcatcgtcttatttccctcaagcggcctgtttacggtgggtgattgtaacgggcataggttaaataaaacttaaagaaagcgtagctatactcgtaataatgtaagaatgtgcttaaccgtggtttcagctacaaaattcgctttctcgttagctgcgcttttattaaactctgcgcgattattattggcgaagaaattgcatgcaaaataaattagct

SEQ ID NO: 15 - DNA, 80 bp, Escherichia coli
aatgatgaaacaggtgagttgagttcaaactgtagtacaattctctccagtttgaacaggaaagaatatgctatgaaccc

SEQ ID NO: 16 - DNA, 165 bp, Escherichia coli
tgccatatgttcgtgaatttacaggcgttagatttacatacatttgtgaatgtatgtaccatagcacgacgataatataaacgcagcaatgggtttattaacttttgaccattgaccaatttgaaatcggacactcgaggtttacatatgaacaaaaacagaggg

SEQ ID NO: 17 - DNA, 366 bp, Escherichia coli
agcgaaggttaatctatcacctaatgtgtatttatacgagaggctaatattgagttgctataaatcgttaaataaataatatatattatttacctaagatacattcactacatcaatatatatttcaatttacgaggttttaattctgcctctttcaacccgcgtcaaaataaaacagtagaatattaatctttttttgtgtttatgtgccttgagatgcctgtattcataactattccttacatcgacgaatgataatttgtaggatagcgaactgtatttttctttctgcgagttaacgcgttgcctttttgggtaaataacgcgcttttggttttttgaggaatagtaatgacgaaacatgcc

SEQ ID NO: 18 - DNA, 172 bp, Escherichia coli
catcccaacactgcttagtgcgctggcctatgggctcgcctggaaagtgatggcgattatataacccacaagaatcatttttctaaaacaatacatttactttatttgtcactgtcgttactatatcggctgaaattaatgaggtcatacccaaatggatagttcgtttacg

SEQ ID NO: 19 - DNA, 276 bp, Escherichia coli
cgttgttatcctgtgtatctgggttatcagcgaaaagtataaggggtaaacaaggataaagtgtcactctttagctagccttgcatcgcattgaacaaaacttgaaccgatttagcaaaacgtggcatcggtcaattcattcatttgacttatacttgcctgggcaatattatcccctgcaactaattacttgccagggcaactaatgtgaaaagtaccagcgatctgttcaatgaaattattccattgggtcgcttaatccatatggttaatcag

SEQ ID NO: 20 - DNA, 126 bp, Escherichia coli
cgcgtcatctcgctcaaaaatccagatttataaaagaaaaaatgactggccagcatcgcaacatgctggcctttttggcaagcaggtcggctcagccgatgagttaagaagatcgtggagaacaat

(19) United States
(12) Patent Application Publication
(10) Pub. No.: US 2003/0186292 A1
(43) Pub. Date: Oct. 2, 2003

(54) Methods for identifying DNA molecules that encode a natural product having bioactivity or encode a protein involved in the production of natural product having bioactivity

(76) Inventors: Ian MacNeil, Milton, MA (US); Choi Lai Yip Tiong, Quincy, MA (US); Kara Brown, Arlington, MA (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/360,201

(22) Filed: Feb. 7, 2003

Related U.S. Application Data
(60) Provisional application No. 60/355,083, filed on Feb. 8, 2002.

(30) Foreign Application Priority Data
Jun. 13, 2002 (GB) 0213616.6

(51) Int. Cl. C12Q 1/68; C12N 15/74; C12N 1/21

(52) U.S. Cl. 435/6; 435/471; 435/252.3

(57) ABSTRACT

Provided herein is a novel and useful method for determining whether an exogenous DNA molecule encodes for a natural product having bioactivity, or a protein having bioactivity. Also provided is a method for separating environmentally derived DNA molecules that encode a natural product having bioactivity or a protein involved in the production of a bioactive molecule from environmentally derived DNA molecules that do not, and a heretofore unknown promoter.

This application claims the benefit of U.S. Provisional Application No. 60/355,083 filed on Feb. 8, 2002 and British Application No. 0213616.6 filed on Jun. 13, 2002, which are hereby incorporated by reference in their entireties.

### FIELD OF THE INVENTION

The present invention relates to heretofore-unknown methods for identifying environmentally derived DNA molecules that encode natural products having bioactivity or proteins involved in the production of such natural products, a heretofore known promoter.

### BACKGROUND OF THE INVENTION

Natural products have been a tremendously rich source of pharmaceutical molecules, accounting for greater than 30% of all human therapeutics and more than 60% of anti-infective and anti-cancer drugs. Despite the advances in high throughput screening technology and attempts to isolate and culture microorganisms from exotic environments, the rate of discovery of novel products has declined. However, it is clear that the vast majority of microorganisms in the environment are still unknown, and that most of them are unculturable under standard laboratory conditions. Since the number of microbial species in the soil that cannot be grown in the laboratory represent at least 98% of the total population, these uncultured species may provide a large, untapped pool of novel natural products. Modern molecular biology offers a way to circumvent this problem: DNA of unculturable organisms can be isolated directly from environmentally derived samples, cloned into suitable vectors, and expressed in a surrogate host that can be grown in the laboratory.

An effort has been made in evaluating such environmentally derived DNA molecules to identify those that encode for a natural product having bioactivity, or a protein involved in the production of a bioactive natural product in a cell. This effort, which is discussed in U.S. Pat. No. 5,824,485 (the '485 patent), utilizes host organisms that are engineered to include a gene encoding a reporter protein operatively associated with a chemoresponsive promoter that responds to the desirable class of metabolites, i.e. natural products, to be detected.

However, the effort described in the '485 patent possess inherent limitations in identifying environmentally-derived DNA molecules that encode for natural products having bioactivity, or proteins involved in the production of such natural products. Initially, the method discussed in the '485 patent is silent with respect to the number of copies of the gene encoding a reporter protein operatively associated with a chemoresponsive promoter that contained within a host cell, as well as the location of such a chimeric DNA molecule within a host cell. It is well known that there are many regulators within the host which control the activity of chemoresponsive promoters, (e.g. a stress promoter). A pre-screen system that utilizes a multiple copy plasmid to insert a chimeric DNA molecule, as discussed above, would result in the presence of multiple copies of the chimeric DNA, and thus multiple copies of the chemoresponsive promoter, in the cell. It is well known the control of the stress response in bacteria is dependent on the intracellular concentration of the various regulators with respect to their gene targets Therefore, a chemoresponsive promoter in a multiple copy system would be unregulated i.e. “on” all the time and be unable to detect a perturbation in homeostasis due to the bioactivity of a natural product.

Accordingly, what is needed is a method for identifying environmentally-derived DNA molecules that encode a natural product having bioactivity or a protein involved in the production of a natural product having bioactivity in which the host cell utilized contains only one copy of reporter regimen that is incorporated into the genome of the host. As a result, detection of the reporter protein in the host cell would only be as a result of bioactivity of the natural product.

Moreover, due to the near limitless number of environmentally-derived DNA molecules, the time needed to identify those that encode natural products having bioactivity, or proteins involved in the production of natural products using heretofore known methods is prohibitive. Accordingly, what is also needed is a quick and efficient way of separating environmentally derived DNA molecules that encode natural products or proteins involved in the production of natural products environmentally derived DNA molecules that do not.

The citation of any reference herein should not be construed as an admission that such reference is available as “Prior Art” to the instant application.

### SUMMARY OF THE INVENTION

Provided herein are novel and useful methods for identifying environmentally derived DNA molecules that encode natural products having bioactivity, or proteins involved in the production of natural products having bioactivity. Such methods do not produce excessive background that could mask positive results. Moreover, methods of the present invention permit the efficient examination of a large amount of environmentally-derived DNA molecules in a short period of time.

Broadly, the present invention extends to a method for identifying a DNA molecule that encodes a natural product having bioactivity or a protein involved in the production of a natural product having bioactivity. The steps of such a method comprise providing a transformed bacterial cell comprising a gene fusion incorporated into the transformed bacterial cell's genome so that only one copy of the gene fusion is present within the transformed bacterial cell, and an exogenous DNA molecule that encodes for the natural product or the protein involved in the production of the natural product within the transformed bacterial cell. The gene fusion comprises a DNA molecule that encodes for a reporter protein operatively associated with a homeostatic promoter which may be activated by the natural product. Other steps of a method of the present invention comprise incubating the transformed bacterial cell under conditions that permit the production of the natural product, and then detecting the presence of the reporter protein within the transformed bacterial cell. The detection of the reporter protein indicates that the natural product produced in the cell caused, either directly or indirectly, the activation of the homeostatic promoter. Consequently, the exogenous DNA molecule encodes a natural product having bioactivity, or a protein involved in the production in the transformed bacterial cell of a natural product having bioactivity.

Numerous types of bacterial cells having applications in a method of the present invention. Examples of such bacterial cells include, but certainly are not limited to E. coli, Bacillus, Staphylococcus, Streptomyces, Myxobacteria and Pseudomonas, to name only a few. A particular example of a bacterial cell having applications herein is E. coli.

Similarly, numerous reporter proteins and the DNA molecules that encode them may be used in the present invention. Examples include green fluorescent protein (GFP), β-galactosidase, luciferase, etc.

Generally, two types of homeostatic promoters may be used in a method of the present invention. One type is a stress promoter, which is defined infra. Particular examples of stress promoters having applications herein include, but certainly are not limited to katG, micF, osmY, uspA, ibpA, rpoH, recA, clpB, gyrA, ada and dinD, to name a few. Another type of homeostatic promoter having applications herein is a multidrug transporter promoter, such as EmrD, MdfA, EmrAB, EmrE, AcrAB, RAB, acrD, acrEF, emrR, etc.

Naturally, numerous methods for transforming a bacterial cell so that it contains an exogenous DNA molecule are readily known to those of ordinary skill in the art and have applications herein. A particular method for inserting the exogenous DNA molecule into a bacterial host, particularly when the exogenous DNA molecule is environmentally-derived, which are generally large pieces of DNA, e.g., about 20 kb to about 300 kb, is via a bacterial artificial chromosome, which is described infra.

Furthermore, the present invention extends to a method for identifying a DNA molecule that encodes a natural product having bioactivity or a protein involved in the production of a natural product having bioactivity, comprising the steps of providing a transformed E. coli cell that comprises a gene fusion incorporated into the genome of the transformed E. coli cell so that only one copy of the gene fusion is present within the transformed E. coli cell, and a bacterial artificial chromosome comprising an exogenous DNA molecule that encodes for the natural product or the protein involved in the production of the natural product within the transformed E. coli cell. The gene fusion comprises a DNA molecule that encodes for a reporter protein operatively associated with a homeostatic promoter which may be activated by the natural product. The transformed E. coli cell is then incubated under conditions that permit the production of the natural product, and the presence of the reporter protein within the transformed E. coli cell is detected. The detection of the reporter protein within the transformed E. coli cell indicates that the exogenous DNA molecule encodes a natural product having bioactivity or a protein involved in the production of a natural product having bioactivity. In a particular embodiment of such a method, the exogenous DNA molecule is an environmentally-derived DNA molecule, the reporting protein is green fluorescent protein, and the detecting step is performed using cytometry, e.g., flow cytometry.

The present invention also extends to a method for identifying an environmentally-derived DNA molecule that encodes a natural product having bioactivity or a protein involved in the production of a natural product having bioactivity, comprising the steps of:

(a) providing a transformed E. coli cell comprising:

(i) a gene fusion incorporated into the transformed E. coli cell's genome so that only one copy of the gene fusion is present within the transformed E. coli cell, wherein the gene fusion comprises a DNA molecule that encodes green fluorescent protein, operatively associated with a homeostatic promoter which may be activated by the natural product to produce the green fluorescent protein, and

(ii) a bacterial artificial chromosome comprising the environmentally-derived DNA molecule that encodes for the natural product or a protein involved in the production of the natural product within the transformed E. coli cell;

(b) incubating the transformed E. coli cell under conditions that permit the production of the natural product; and

(c) detecting with cytometry the green fluorescent protein within the transformed E. coli cell.

The detection of the green fluorescent protein within the transformed E. coli cell indicates that the environmentally-derived DNA molecule encodes a natural product having bioactivity or a protein involved in the production of a natural product having bioactivity.

In another embodiment, the present invention extends to a method for separating environmentally-derived DNA molecules that encode natural products having bioactivity or proteins involved in the production of natural products having bioactivity, from environmentally-derived DNA molecules that do not encode natural products having bioactivity or proteins involved in the production of natural products having bioactivity. Once this separation occurs, the environmentally derived DNA molecules that are involved either directly or indirectly in the production of bioactive natural products can be collected for form a “high quality” library, i.e., a library of environmentally derived DNA molecules that can undergo a detailed analysis or can be transferred to another host strain. Moreover, it is a simple matter to add environmentally derived DNA molecules involved in the production of bioactive natural products. Such a method of the present invention comprises initially providing transformed bacterial cells wherein each transformed bacterial cell comprises a gene fusion incorporated into the transformed bacterial cell's genome so that only one copy of the gene fusion is present within each transformed bacterial cell. The gene fusion comprises a DNA molecule that encodes for a reporter protein operatively associated with a homeostatic promoter which may be activated by a natural product. The homeostatic promoter used in each gene fusion can be the same or different. Then, each transformed bacterial cell is transformed with a bacterial artificial chromosome comprising an environmentally-derived DNA molecule that encodes for a natural product or a protein involved in the production of a natural product so that each bacterial artificial chromosome comprises only one environmentally-derived DNA molecule. Naturally, to maximize the number of environmentally derived DNA molecules to be examined, each bacterial artificial chromosome may contain a different environmentally derived DNA molecule. The transformed bacterial cells are then incubated under conditions that permit the production of the natural products. If the natural product possesses bioactivity, it will activate the homeostatic promoter so that the cell produces the reporter protein. Those cells in which the reporter protein is detected are then separated from the transformed bacterial cells in which no reporter protein is detected. The separated transformed bacterial cells in which the reporter protein is detected contain the environmentally-derived DNA molecules that encode natural products having bioactivity, or proteins involved in the production of natural products having bioactivity. Thus, the environmentally derived DNA molecules that encode a natural product having bioactivity or a protein involved in the production of a natural product having bioactivity are separated from the environmentally derived DNA molecules that do not encode a natural product having bioactivity or a protein involved in the production of such a natural product.

Numerous bacterial cells have applications in a method of the present invention. Particular examples include, but certainly are not limited to E. coli, Bacillus Staphylococcus, Streptomyces, Myxobacteria and Pseudomonas, to name only a few. A particular bacterial cell having applications herein is E. coli.

Likewise, numerous homeostatic promoters have applications in a method of the present invention. Examples of such promoters include stress promoters, such as, for example, katG, micF, osmY, uspA, ibpA, rpoH, recA, clpB, gyrA, ada, dinD or multidrug transporter promoters, such as EmrD, MdfA, EmrAB, EmrE, AcrAB, RAB, acrD, acrEF, emrR, etc.

Similarly, numerous reporter proteins may be used in a method of the present invention. Examples include, but certainly are not limited to green fluorescent protein, β-galactosidase, or luciferase. In a particular example of a method of the present invention for separating environmentally derived DNA molecules, the reporter protein is green florescent protein, and the separating step is performed with flow cytometry.

Optionally, a method of the present invention for separating environmentally derived DNA molecules that encode natural products or proteins involved in the production of natural products having bioactivity may be performed in a high throughput method as described infra.

Furthermore, the present invention extends to a method for separating environmentally-derived DNA molecules that encode natural products having bioactivity or proteins involved in the production of natural products having bioactivity from environmentally-derived DNA molecules that do not encode natural products having bioactivity or proteins involved in the production of natural products having bioactivity. Such a method comprises the steps of:

(a) providing transformed E. coli cells comprising a gene fusion incorporated into the genome of each transformed E. coli cell so that only one copy of the gene fusion is present within each transformed E. coli cell, the gene fusion comprising a DNA molecule that encodes green fluorescent protein, operatively associated with a homeostatic promoter which may be activated by a natural product,

(b) transforming each transformed E. coli cell with a bacterial artificial chromosome comprising an environmentally-derived DNA molecule that encodes the natural product or a protein involved in the production of the natural product, so that each bacterial artificial chromosome comprises only one environmentally-derived DNA molecule;

(c) incubating the transformed E. coli cells under conditions that permit the production of the natural products; and

(d) separating the transformed E. coli. cells in which the green fluorescent protein is detected from the transformed E. coli. cells in which the green fluorescent protein is not detected.

The separated transformed E. coli cells in which the green fluorescent protein is detected contain the environmentally-derived DNA molecules that encode natural products having bioactivity, or proteins involved in the production of natural products having bioactivity.

Furthermore, the present invention extends to a heretofore unknown promoter for controlling the expression of a DNA molecule that encodes an amino acid sequence. The promoter has a DNA sequence consisting essentially of:

(SEQ ID NO:20)

CGCGTCATCTCGCTCAAAAATCCAGATTTATAAAAGAAAAAATGACTGGC
CAGCATCGCAACATGCTGGCCTTTTTGGCAAGCAGGTCGGCTCAGCCGAT
GAGTTAAGAAGATCGTGGAGAACAAT.

Exposure of a host cell, containing this promoter operatively-associated with a reporter gene, to an organic solvent, such as ethanol, activates the promoter causing expression of the reporter gene.

Furthermore, the present invention extends to a method for identifying an environmentally derived DNA molecule that encodes a natural product that inhibits a homeostatic response or a protein involved in the production of a natural product that inhibits a homeostatic response. The first step of such a method is to provide a transformed bacterial cell comprising a gene fusion incorporated into the genome of the transformed bacterial cell so that only one copy of the gene fusion is present within the transformed bacterial cell. Such a gene fusion comprises a DNA molecule that encodes a reporter protein operatively associated with a homeostatic promoter. Then, the transformed bacterial cell is transformed with a bacterial artificial chromosome comprising an environmentally-derived DNA molecule that encodes a natural product or a protein involved in the production of the natural product. The cell is then incubated under conditions that permit the production of the natural product. Subsequently, the cell is contacted with a known inducer of the homeostatic promoter in order to activate the cell, which is then examined for the presence of the reporter protein. A lack of detection of the reporter protein indicates that the environmentally derived DNA molecule encodes a natural product or protein involved in the production of a natural product that inhibits the normal response to alterations in homeostasis. Naturally E. coli, Bacillus Staphylococcus, Streptomyces, Myxobacteria and Pseudomonas cells have applications in such a method. Moreover, the different types of reporter proteins and homeostatic promoters have applications in such a method. What's more, such a method may optionally be performed in a high throughput fashion as described infra.

Accordingly, it is an aspect of the present invention to provide a novel and useful method for identifying environmentally derived DNA molecules that encode natural products having bioactivity or proteins involved in the production of natural products having bioactivity from environmentally derived DNA molecules that do not encode natural products having bioactivity or proteins involved in the production of natural products having bioactivity.

It is another aspect of the present invention to provide a method for separating environmentally derived DNA molecules that encode natural products having bioactivity or proteins involved in the production of natural products having bioactivity from environmentally derived DNA molecules that do not. Once the separation has occurred, the environmentally derived DNA molecules that encode for a bioactive natural product or a protein involved in the production of bioactive natural protein can be collected and place in a “high quality” library to screen various compounds for agonist or antagonist activity of a bioactive natural product.

It is still another embodiment to provide a heretofore unknown promoter having an activity that is readily controllable. Consequently, the expression of a DNA molecule operatively associated with the promoter of the present invention can be readily controlled.

These and other aspects of the present invention will be better appreciated by reference to the following drawings and Detailed Description.

### BRIEF DESCRIPTION OF THE DRAWINGS

FIG. 1: A schematical view of a method of the present invention for separating environmentally derived DNA molecules that encode a natural product having bioactivity or a protein involved in the production of a natural product having bioactivity from environmentally derived DNA molecules that do not have bioactivity.

FIG. 2: Panel A:-A histogram of 10⁶ bacteria cells containing the novel promoter of the present invention fused to GFPuv. Positive cells were selected from the area under region M1. Panel B-A histogram of an individual positive clone (open line) compared to vector alone (filled line).

FIG. 3: Panel A-A schematical view of a promoter of the present invention. Panel B-DNA sequence of the promoter of the present invention showing the fusion of 4 amino acids of the native protein (EmrA) and GFPuv.

### DETAILED DESCRIPTION OF THE INVENTION

As explained above, the present invention broadly extends to a method for identifying a DNA molecule that encodes a natural product having bioactivity or a protein involved in the production of a natural product having bioactivity, comprising the steps of:

a) providing a transformed bacterial cell comprising:

(i) a gene fusion incorporated into the transformed bacterial cell's genome so that only one copy of the gene fusion is present within the transformed bacterial cell, wherein the gene fusion comprises a DNA molecule that encodes for a reporter protein operatively associated with a homeostatic promoter which may be activated by the natural product, and

(ii) an exogenous DNA molecule that encodes for the natural product or the protein involved in the production of the natural product within the transformed bacterial cell;

(b) incubating the transformed bacterial cell under conditions that permit the production of the natural product; and

(c) detecting the presence of the reporter protein within the transformed bacterial cell.

The detection of the reporter protein within the transformed bacterial cell indicates that the exogenous DNA molecule encodes a natural product having bioactivity or a protein involved in the production of a natural product having bioactivity. In a particular embodiment, the exogenous DNA molecule is an environmentally derived DNA molecule.

The present invention is based upon the discovery that, surprisingly and unexpectedly, transformed bacterial hosts that comprise a gene fusion comprising a reporter gene operatively associated with a homeostatic promoter such that only one copy of the gene fusion is present within the cell and incorporated into the cell's genome, provide a sensitive and accurate bioassay for identifying exogenous DNA molecules that encode a natural product or a protein involved in the production of a natural product within the cell. In particular, the present invention utilizes the myriad of responses which exist in a host organism for dealing with stress brought on by many environmental and chemical stimuli, including a natural product not normally present in the cell. When the host cell produces the natural product, the cell's response is to activate one or many stress response genes and/or proteins involved in multidrug transporter promoters (MDT). Homeostatic promoters regulate these stress response genes and multidrug transporter promoters within the cell. Thus, in a method of the present invention, wherein a DNA molecule encoding a reporter protein is operatively associated with a homeostatic promoter, detection of the reporter protein in the transformed bacterial cell indicates that the exogenous DNA, e.g., environmentally derived DNA, encodes a natural product or a protein involved in the production of a bioactive natural product that interacts with the homeostatic promoter, and consequently possesses bioactivity.

Furthermore, the present invention also extends to a method for separating environmentally derived DNA molecules that encode for a natural product having bioactivity or a protein involved in the production of a natural product having bioactivity. Such a method comprises the steps of:

(a) providing transformed bacterial cells wherein each transformed bacterial cell comprises a gene fusion incorporated into the transformed bacterial cell's genome so that only one copy of the gene fusion is present within each transformed bacterial cell, the gene fusion comprising a DNA molecule that encodes for a reporter protein operatively associated with a homeostatic promoter which may be activated by a natural product;

(b) transforming each transformed bacterial cell with a bacterial artificial chromosome comprising an environmentally-derived DNA molecule that encodes for the natural product or a protein involved in the production of the natural product so that each bacterial artificial chromosome comprises only one environmentally-derived DNA molecule;

(c) incubating the cells under conditions that permit the production of the natural products; and

(d) separating the transformed bacterial cells in which the reporter protein is detected from the transformed bacterial cells in which the reporter protein is not detected.

The separated transformed bacterial cells in which the reporter protein is detected contain the environmentally-derived DNA molecules that encode natural products having bioactivity, or proteins involved in the production of natural products having bioactivity. Consequently separating these cells from cells that do not produce a bioactive natural product separates environmentally derived DNA molecules that encode natural products having bioactivity or proteins involved in the production of natural products having bioactivity from environmentally derived DNA molecules that do not.

Numerous terms and phrases are used throughout the instant specification and claims. Accordingly, as used herein, the term “natural product” refers to a product produced directly from something found in nature, with little or no modification. Examples of natural products include, but certainly are not limited to proteins, nucleic acid molecules, carbohydrates, metabolites, etc.

As used herein, the phrase “protein involved in the production of a natural product” refers to protein that causes a chemical reaction within a cell or catalyzes a chemical reaction within a cell so that a natural product can be synthesized within the cell. A particular example of such a protein is an enzyme.

As used herein, the term “homeostasis” refers to the maintenance of a stable internal cellular environment.

As used herein, the term “bacterial artificial chromosome” refers to a vector used to clone DNA fragments (20-300 kb insert size) in bacterial cells. It is based on the naturally occurring F-factor plasmid found in the bacterium E. coli.

As used herein, the term “environmentally derived DNA molecule” refers to DNA from environmental organisms (e.g. bacteria) isolated directly from the environment (e.g. soil, water, etc.)

Furthermore, in accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control. A bacterial artificial chromosome is a type of vector. A BAC may be introduced into a host cell by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

A “cassette” refers to a segment of DNA that can be inserted into a vector at specific restriction sites. The cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Exogenous” DNA refers to DNA not naturally located in the cell, or in a chromosomal site of the cell. A particular example of an exogenous DNA molecule is an environmentally derived DNA molecule.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

“Homologous recombination” refers to the insertion of a foreign DNA sequence of a vector into a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination. Homologous recombination may be used to insert a gene fusion into the genome of a transformed bacterial cell in a method of the present invention.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences.

Transcriptional and translational control sequences are DNA regulatory sequences, such as promoters, enhancers, terminators, and the like, that provide for the expression of a coding sequence in a host cell.

The terms “promoter sequence” and “promoter” can be used interchangeably, and refer to a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. A DNA sequence is “operatively associated” to an expression control sequence, such as a promoter, when the expression control sequence controls and regulates the transcription and translation of that DNA sequence. The term “operatively associated” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If an isolated nucleic acid molecule to be expressed does not contain an appropriate start signal, such a start signal can be inserted into the expression vector in front of (5′ of) the isolated nucleic acid molecule.

As used herein, the term “homeostatic promoter” refers to a promoter that regulates a response gene in response to the presence of a natural product in the cell. Generally, there are two types of homeostatic promoters: stress promoters and multidrug transporter promoters (MDT). Naturally, both of these types of promoters have applications in a method of the present invention. Particular stress promoters and their DNA sequences that may be used in a method of the present invention are discussed below:

Stress Promoters

katG

The concentration of hydrogen peroxide in the bacterial cell is controlled through the action of catalases and peroxidases, collectively called hydroperoxidases. Hydrogen peroxide causes direct DNA damage through the occurrence of single-strand breaks. In E. coli there are three hydroperoxidases, HPI, HPII, and HPIII. Intracellular sources of hydrogen peroxide include photodegredation products of near-ultraviolet radiation, as well as being a product of superoxide-dismutase-mediated catalysis of the superoxide anion, O²⁻. The katG gene encodes hydrogen peroxidase I, a tetrameric bifunctional catalase and o-dianiside . KatG responds to oxidative damage and is induced by hydrogen peroxide. It has a DNA sequence of

(SEQ ID NO:1)

GTGTGGCTTTTGTGAAAATCACACAGTGATCACAAATTTTAAACAGAGCA
CAAAATGCTGCCTCGAAATGAGGGCGGGAAAATAAGGTTATCAGCCTTGT
TTTCTCCCTCATTACTTGAAGGATATGAAGCTAAAACCCTTTTTTATAAA
GCATTTGTCCGAATTCGGACATAATCAAAAAAGCTTAATTAAGATCAATT
TGATCTACATCTCTTTAACCAACAATATGTAAGATCTCAACTATCGCATC
CGTGGATTAATTCAATTATAACTTCTCTCTAACGCTGTGTATCGTAACGG
TAACACTGTAGAGGGGAGCACATTGATGAGCACGTCA.

MicF

The major outer membrane proteins of E. coli are OmpF and OmpC. As the osmolarity of the culture medium increases, the OmpF production decreases while OmpC increases, maintaining the same amount of Omp proteins in the outer membrane. The regulation of the translation of OmpF is in part mediated by a short antisense RNA which is the complement to the 5′ end of the OmpF mRNA. This antisense molecule is micF, which is short for mRNA-interfering complementary RNA. The production of mic RNA is in proportion to the level of ompC mRNA, creating an efficient way to maintain a constant level of Omp proteins in the outer membrane. The ompF and ompC genes are also positively regulated by OmpR. micF responds to fluctuations in temperature and osmotic conditions, as well as a wide range of chemicals that interfere with the integrity of the outer membrane, including p-chloroanaline, dimethyl sulfoxide (DMSO), ethanol, nalidixic acid, and phenol. It has a DNA sequence of

(SEQ ID NO:2)

GACCTGTTAAAACTTCGCGAAGTGATAGGGCAGTATAAAGGGTACAGTGT
GAAATTCCAGACTCTTACCTGATTGATGATTCTCTATTCTCCCGTGATAT
TTATCATCCTGCATTGGGGGAAATAGATGTACAAGCGCCATTTTGCTGTA
CAAATAACCTACAAAAAGCCCAACAAAAATAATTGCAAATAAACAAAGAT
TGCTGGAAATTATGCGGATGTTAATTATTTGTGAAATAGTTAACAAGCGT
TATAGTTTTTCTGTGGTAGCACAGAATAATGAAAAGTGTGTAAAGAAGGG
TAAAAAAAACCGAATGCGAGGCATCCGGTTGAAATAGGGGTAAACAGACA
TTCAGAAATGAATGACGGTAATAAATAAAGTTAATGATGATAGCGGGAGT
TATTCTAGTTGCGAGTGAAGGTTTTGTTTTGACATTCAGTGCTGTCAAAT
ACTTAAGAATAAGTTATTGATTTTAACCTTGAATTATTATTGCTTGATGT
TAGGTGCTTATTTCGCCATTCCGCAATAATCTTAAAAAGTTCCCTTGCAT
TTACATTTTGAAACATCTATAGCGATAAATGAAACATCTTAAAAGTTTTA
GTATCATATTCGTGTTGGATTATTCTGCATTTTTGGGGAGAATGGACTTG
CCGACTGATTAATGAGGGTTAATCAGTATGCAGTGGCATAAAAAAGCAAA
TAAAGGCATATAACAGAGGGTTAATAACATGAAAGTTAAA.

osmY

This is a periplasmic protein induced by both changes in growth conditions and osmotic conditions. The osmY gene is an rpoS-dependent gene. The rpoS gene encodes an alternate sigma factor for RNA polymerase. osmY responds to compounds such as sodium nitrite, ethanol, and nalidixic acid. The osmY promoter has a DNA sequence of

(SEQ ID NO:3)

TGGCACAGGAACGTTATCCGGACGTTCAGTTCCACCAGACCCGCGAGCAT
TAATTCTTGCCTCCAGGGCGCGGTACGCGCTGCGCCCTGTCAATTTCCCT
TCCTTATTAGCCGCTTACGGAATGTTCTTAAAACATTCACTTTTGCTTAT
GTTTTCGCTGATATCCCGAGCGGTTTCAAAATTGTGATCTATATTTAACA
AAGTGATGACATTTCTGACGGCGTTAAATACCGTTCAATGCGTAGATATC
AGTATCTAAAGCCGTCGATTGTCATTCTACCGATATTAATAACTGATTCA
GAGGCTGTAATGGTCGTTATTCATCACTCATCGCTTTTGTGATGGCGACC
ATTGACTTCTGTAGAGGGTGAAGTCTCTCCCTATTCAGCAATGCAACCTC
GTGTTGCCAGGCTCAAATTACGAGCAAACATACAGGAATAAATCGATGAC
TATGACA.

uspA

The level of universal stress protein is increased in the cytoplasm of E. coli under conditions of growth inhibition. The growth inhibiting conditions which induced the presence of this protein include nutrient exhaustion of carbon, nitrogen, phosphate, or sulfate, as well as the presence of toxic agents like heavy metals, oxidants, acids, and antibiotics. uspA responds to DNA damaging compounds such as 4-nitroquiloline 1-oxide (4-NQO) and nalidixic acid, as well as chemicals like propanol. The uspA promoter has a DNA sequence of

(SEQ ID NO:4)

CCCGCGGAGTTCCACCCCGGGGCTACCGCTCCCGATACGCTGCCAATCAG
TTAACACCAGGTCCTGGAGAAACCGCTTTTGTGGTGACCAACATACGAGC
GGCTCTATAGATAGTGTAGGAGATCAGGTTGTTTTTTTTCCAGAAGGTTA
ACCACTATCAATATATTCATGTCGAAAATTTGTTTATCTAACGAGTAAGC
AAGGCGGATTGACGGATCATCCGGGTCGCTATAAGGTAAGGATGGTCTTA
ACACTGAATCTTTACGGCTGGGTTAGCCCCGCGCACGTAGTTCGCAGGAC
GCGGGTGACGTAACGGCACAAGAAACGCTAGCTGGCCAGTCATCGACAAC
TTTATGGAAGGAGTAACACTATGGCTTATAAACAC.

ibpA

The ibp operon encodes the heat shock proteins ibpA and ibpB which are induced following a temperature upshift from 37° C. to 42° C. The ibpA promoter has a DNA sequence of

(SEQ ID NO:5)

TACGTCGCACTGTGGCGGCTATCGCACTTTAACGTTTCGTGCTGCCCCCT
CAGTCTATGCAATAGACCATAAACTGCAAAAAAAAGTCCGCTGATAAGGC
TTGAAAAGTTCATTTCCAGACCCATTTTTACATCGTAGCCGATGAGGACG
CGCCTGATGGGTGTTCTGGCTACCTGACCTGTCCATTGTGGAAGGTCTTA
CATTCTCGCTGATTTCAGGAGCTATTGATT.

cspA

This is the major cold shock response protein in E. coli. Under cold shock conditions (10° C.), approx. 10% of the synthetic machinery is dedicated to this response. The cspA promoter has a DNA sequence of

(SEQ ID NO:6)

ACTTTTATCCACTTTATTGCTGTTTACGGTCCTGATGACAGGACCGTTTT
CCAACCGATTAATCATAAATATGAAAAATAATTGTTGCATCACCCGCCAA
TGCGTGGCTTAATGCACATCAACGGTTTGACGTACAGACCATTAAAGCAG
TGTAGTAAGGCAAGTCCCTTCAAGAGTTATCGTTGATACCCCTCGTAGTG
CACATTCCTTTAACGCTTCAAAATCTGTAAAGCACGCCATATCGCCGAAA
GGCACACTTAATTATTAAAGGTAATACACT.

rpoH

The P3 promoter of the rpoH gene (sigma 32) is one of five promoters known to be activated by protein misfolding. This factor is essential for the transcription of heat-shock genes. The rpoH promoter has a DNA sequence of

(SEQ ID NO:7)

TAAAAGCGTGTTATACTCTTTCCCTGCAATGGGTTCCGTAGCAGGGAAAG
AGACCCCGTTGTCTCTTCCCGGTATTTCATCTCTATGTCACATTTTGTGC
GTAATTTATTCACAAGCTTGCATTGAACTTGTGGATAAAATCACGGTCTG
ATAAAACAGTGAATGATAACCTCGTTGCTCTTAAGCTCTGGCACAGTTGT
TGCTACCACTGAAGCGCCAGAAGATATCGATTGAGAGGATTTGAATGACT
GACAAAATG.

recA

In enteric bacteria, agents which cause DNA damage or block DNA replication induce an extensive regulon called the SOS response. The genes that make up this regulon are all negatively regulated by the LexA repressor. The recA gene encodes a protein which is a key member of the SOS response regulon. RecA is involved in DNA repair and recombination. The inducibility of recA under the SOS response is mediated through the proteolytic cleavage of the LexA repressor by an activated-RecA protein. Therefore, a fairly high basal level of RecA exists in the cell for processes not immediately involved in the SOS response but required for activation of RecA. RecA responds to DNA damage as induced by chemicals like methyl methanesulfonate (MMS) and mitomycin C. The recA promoter has a DNA sequence of

(SEQ ID NO:8)

ACTTGATACTGTATGAGCATACAGTATAATTGCTTCAACAGAACATATTG
ACTATCCGGTATTACCCGGCATGACAGGAGTAAAAATGGCTATCGACGAA
AACAAACAGAAAGCG.

clpB

Induction of the heat shock response occurs coordinately at the level of transcription in part due to the presence of an RNA polymerase heat shock sigma factor 32 in E. coli. Among the genes in the heat shock regulon are two ATP-dependent proteases, Lon and Clp. Clp consists of a regulatory subunit (ClpA) and a proteolytic subunit (ClpP). In addition, there is an analog of the CIpA protein named ClpB. Unlike clpA, which is not controlled by sigma 32, the clpB gene is under sigma 32 transcriptional control. Therefore, ClpB is involved in the proteolytic activation of ClpP following cellular stress. clpB responds to agents which affect proteins, such as acetone, chloroform, methapyrilene, and p-chloroaniline. The clpB promoter has a DNA sequence of

(SEQ ID NO:9)

CTTTTCACATTAATCTGGTCAATAACCTTGAATAATTGAGGGATGACCTC
ATTTAATCTCCAGTAGCAACTTTGATCCGTTATGGGAGGAGTTATGCGTC
TG.

dinD

Following the occurrence of extensive DNA damage to the E. coli genome, the SOS response becomes activated (see RecA above). The goal of this SOS response is to minimize the impact of the incurred DNA lesions. The genes involved in this DNA-damage inducible response encode enzymes required for the processing of damaged DNA, including DNA repair enzymes and enzymes involved in DNA recombination. Another set of less well-characterized genes that are induced by damaged DNA are collectively referred to as the din genes, short for DNA damage inducible. dinD responds to many different types of DNA damaging chemicals like mitomycin C, methyl methanesulfonate (MMS), methyl-nitro-nitrosoguanidine (MNNG), nalidixic acid, b-napthoflavone, 4-nitroquinoline 1-oxide (4-NQO). The dinD promoter has a DNA sequence of

| | |
|---|---|
| ATAACTCGTAACGCCAATTCTTACTTTTCCGCCTTCACAAATGCCGCCACTCAAACAGAGC | (SEQ ID NO:10) |
| GGCATTTTTCTTCCCCGCAACATTCAATTCTGTTTTGCGTGCCTGCTCCAGATTTTGCGATG | |
| TTTTTTTGCCCAGCACACTGAGAACGTGAGATACTCACAACTGTATATAAATACAGTTACA | |
| GATTTACTTTCTTTGCAATTGATATCACATGGAGTGGGCA. | |

gyrA

The gyraseA stress promoter. DNA gyrase is the bacterial enzyme that introduces negative supercoils into DNA. The two subunits of gyrase are encoded by gyrA and gyrB. Agents which affect the topology of DNA activate the gyrase locus. More importantly, inhibitors of gyrase function cause the activation of gyrase locus via the activation of the gyrA promoter. Inhibitors of gyrase, such as the quinolones, are clinically effective antibiotics. The gyraseA stress promoter has a DNA sequence of

| | |
|---|---|
| ATTGGCACTTCTACTCCGTAATTGGCAAGACAAACGAGTATATCAGGCATTGGATGTGAA | (SEQ ID NO:11) |
| TAAAGCGTATAGGTTTACCTCAAACTGCGCGGCTGTGTTATAATTTGCGACCTTTGAATCC | |
| TGGGATACAGTAGAGGGATAGCGGTTAG | |

ada

Alkylating agents are toxic and mutagenic in bacterial systems and carcinogenic to mammalian cells. The ada gene is the regulatory gene and also serves as one of the structural genes of the adaptive response to alkylation damage in E. coli. Following DNA alkylation, the Ada protein transfers the methyl group from O-alkylated lesions in DNA to one of two cysteine residues itself. This self methylation causes the Ada protein to become a strong activator of its own transcription and also of alkA. The ada promoter responds to the presence of methyl adducts on dsDNA. Therefore, the gene is induced by DNA damaging agents like methyl methane sulfonate (MMS), methyl-nitro-nitrosoguanidine (MNNG), and p-chloroaniline. The ada promoter has a DNA sequence of

| | |
|---|---|
| AAGATTGTTGGTTTTTGCGTGATGGTGACCGGGCAGCCTAAAGGCTATCCTTAACCAGGG | (SEQ ID NO:12) |
| AGCTGATTATGAAAAAAGCC. | |

### Multidrug Transporter Promoters

The Major Facilitator Superfamily

The major facilitator superfamily is made of membrane proteins involved in the transport of various molecules including sugars, Kreb cycle intermediates, phosphate esters and antibiotics. The family can be broken down into 2 main groups based on the number of transmembrane segments (TMS).

EmrD and MdfA

EmrD and Mdfa are members of the 12-TMS group. The former is involved in the protection from energy uncouplers and the latter is involved in the efflux of chloramphenicol (10 ug/ml) as well as a wide range of unrelated neutral and positively charged drugs. EmrD and MdfA have DNA sequences of

| | |
|---|---|
| CTTGTTGGTTTTGTGTTTAACAATATTTATACAAGCACAGCTTTACAGGGGAGACAATGGA | (SEQ ID NO:13) |
| AAATTTTTCAGCAAGGGAAAATTGAGGGGTTGATCACGTTTTGTACTGAATTGCAGATAA | |
| CAAAAAACCCCGCCGGAGCGAGGTTTCGTCAGTCGCCTGCGGCTGGTAACCGCAAAGCAC | |
| ACTGTATTATGTCAACACTGAAAGTATACGTGTTCCGCGCAGAACGCGCAATTTCGGCAC | |
| GAATTTTGACGTATTTAGTGCATAGTTGAGTATCGATCACAGTTTGCGTTTTGTCCAAATA | |
| TTACTGTTTATTTATACAGTAAACTTCTATAATATCACTGTACGCAATGTGTTATGCGGGG | |
| GCCGCATCGTTACCCGGCGCACTAAGTCCTGGCTGAAACGGGTGGTGCCGTCAGCGCCTT | |
| AACCCCGCGTGAGCACACTGTGTTATGTCAACAAGCACAACGTTTCTCCTTGAGATACCG | |
| CGTGCACAACAGCTGGCAACAGGCAGCGGAAAGGTACGTCAGCTGGCAGTGCTCCTGAA | |
| CCACAGGAGACGCGTATGAACCTGGTGGATATCGCCATTCTTATCCTCAAACTCATTGTTG | |
| CAGCACTGCAACTGCTTGATGCTGTTCTGAAATACCTGAAGTAATTCAGATTCAAGTCGC | |
| ACCAAAGGGGAGCGGGAAACCGCTCCCCTTTTATATTTAGCGTGCGGGTTGGTGTCGGAT | |
| GCGATGCTGACGCATCTTATCCGCCCTACCATCTCTCCCGGCAACATTTATTGCCGCTTTT | |
| GTTTACATATTCTGCCGCTAAACAATTCCCCATTCCTGGCGTATATCTGGCTAACATTCAT | |
| CAATGTGATAGATTCCTCTCCCGCATTTATGGGAATGCGTAGTGACTTATTCTAATTATTT | |
| TTATAAAAGCATCCGTGATAATGAAAAGGCAA, | |
| and | |
| CCAGCATAACTTCCCGACGCAAAGTGATTAAAAGGGAGCCAATACAGGCAAGTCGTTGA | (SEQ ID NO:14) |
| GAATAAAGTGCAGGTTAAACTGGGTAAAGCGGCATCGTCTTATTTCCCTCAAGCGGCCTG | |
| TTTACGGTGGGTGATTGTAACGGGCATAGGTTAAATAAAACTTAAAGAAAGCGTAGCTAT | |
| ACTCGTAATAATGTAAGAATGTGCTTAACCGTGGTTTCAGCTACAAAATTCGCTTTCTCGT | |
| TAGCTGCGCTTTTATTAAACTCTGCGCGATTATTATTGGCGAAGAAATTGCATGCAAAATA | |
| AATTAGCT, respectively. | |

Small Multi-drug Resistance Family

This family is comprised of short (˜100 aa) proteins which function as homooligeric complexes

EmrE

EmrE confers resistance to ethidium bromide. It has a DNA sequence of

| | |
|---|---|
| AATGATGAAACAGGTGAGTTGAGTTCAAACTGTAGTACAATTCTCTCCAGTTTGAACAGG | (SEQ ID NO:15) |
| AAAGAATATGCTATGAACCC. | |

Resistance Nodulation Cell Division Family

This family of transporters interacts with a membrane fusion protein and an outer membrane protein to allow drug transport across both inner and outer membranes of gram-negative bacteria.

AcrAB

Confers resistance to basic dyes, detergents and antibiotics. It has a DNA sequence of

| | |
|---|---|
| TGCCATATGTTCGTGAATTTACAGGCGTTAGATTTACATACATTTGTGAATGTATGTACCA | (SEQ ID NO:16) |
| TAGCACGACGATAATATAAACGCAGCAATGGGTTTATTAACTTTTGACCATTGACCAATTT | |
| GAAATCGGACACTCGAGGTTTACATATGAACAAAAACAGAGGG. | |

AcrEF

It has a DNA sequence of

| | |
|---|---|
| AGCGAAGGTTAATCTATCACCTAATGTGTATTTATACGAGAGGCTAATATTGAGTTGCTAT | (SEQ ID NO:17) |
| AAATCGTTAAATAAATAATATATATTATTTACCTAAGATACATTCACTACATCAATATATA | |
| TTTCAATTTACGAGGTTTTAATTCTGCCTCTTTCAACCCGCGTCAAAATAAAACAGTAGAA | |
| TATTAATCTTTTTTTGTGTTTATGTGCCTTGAGATGCCTGTATTCATAACTATTCCTTACAT | |
| CGACGAATGATAATTTGTAGGATAGCGAACTGTATTTTTCTTTCTGCGAGTTAACGCGTTG | |
| CCTTTTTGGGTAAATAACGCGCTTTTGGTTTTTTGAGGAATAGTAATGACGAAACATGCC | |

emrR

It has a DNA sequence of

| | |
|---|---|
| CATCCCAACACTGCTTAGTGCGCTGGCCTATGGGCTCGCCTGGAAAGTGATGGCGATTAT | (SEQ ID NO:18) |
| ATAACCCACAAGAATCATTTTTCTAAAACAATACATTTACTTTATTTGTCACTGTCGTTAC | |
| TATATCGGCTGAAATTAATGAGGTCATACCCAAATGGATAGTTCGTTTACG | |

marAB

It has a DNA sequence of

| | |
|---|---|
| CGTTGTTATCCTGTGTATCTGGGTTATCAGCGAAAAGTATAAGGGGTAAACAAGGATAAA | (SEQ ID NO:19) |
| GTGTCACTCTTTAGCTAGCCTTGCATCGCATTGAACAAAACTTGAACCGATTTAGCAAAAC | |
| GTGGCATCGGTCAATTCATTCATTTGACTTATACTTGCCTGGGCAATATTATCCCCTGCAA | |
| CTAATTACTTGCCAGGGCAACTAATGTGAAAAGTACCAGCGATCTGTTCAATGAAATTAT | |
| TCCATTGGGTCGCTTAATCCATATGGTTAATCAG. | |

### High Throughput Screening

As explained above, a method of the present invention, particularly a method for separating environmentally derived DNA molecules that encode bioactive natural products or proteins involved in the production of bioactive natural products from environmentally derived DNA molecules that do not encode such natural products or proteins, can readily be used in a high throughput fashion. High throughput screening systems are commercially available (see, e.g., Zymark Corp., Hopkinton, Ma.; Air Technical Industries, Mentor, Ohio; Beckman Instruments, Inc. Fullerton, Calif.; Precision Systems, Inc., Natick, Ma., etc.) that may be used in a high throughput method of the present invention. These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

### Novel Promoter of the Present Invention

As explained above, the present invention extends to a novel promoter along with a method of regulating the expression of a DNA molecule with the novel promoter of the present invention.

One of the multidrug transporter promoters having applications in a method of the present invention is EmrAB. The EmrAB locus consists of 3 genes- EmrR, EmrA and EmrB. The promoter for this locus, which has well-defined promoter elements, is upstream of the regulator gene (EmrR), which is able to bind to the promoter region and thus, regulate transcription. When the correct class of drug is present, it binds to the EmrR protein, removing it from the promoter region. Hence, transcription of the AB locus is able to proceed. The AB proteins are the structural pump proteins which are necessary for transporting the drug out of the cell.

It has been discovered that, surprisingly an unexpectedly, the DNA sequence between EmrR and the EmrA genes of the locus, which has a DNA sequence of CGCGTCATCTCGCTCAAAAATCCAGATTTATAAAAGAAAAAATGACTGGCCAGCATCGCA ACATGCTGGCCTTTTTGGCAAGCAGGTCGGCTCAGCCGATGAGTTAAGAAGATCGTGGAG AACAAT (SEQ ID NO:20) functions as a promoter in terms of reporter activation, even though it lacks easily identifiable promoter elements. Moreover, organic solvents, such as ethanol, can induce the promoter like activity of this DNA sequence.

The present invention may be better understood by reference to the following non-limiting Examples, which are provided as exemplary of the invention. The following Examples are presented in order to more fully illustrate the present invention. They should in no way be construed, however, as limiting the broad scope of the invention.

### EXAMPLE I

### Method for Identifying Environmentally Derived DNA Molecules that Encode for Natural Products

Materials and Method

Integration To construct strains containing a single copy of the reporter and homeostatic promoter, a system developed by Boyd (Boyd, et al. J. Bact., 2000) for reversible, stable integration of plasmid-borne genes into the Escherichia coli chromosome was used. Using this method, most ordinary E. coli strains and a variety of pBR322-derived ampicillin-resistant plasmids can be used. A single genetic element, a lambda phage, is the only specialized vector required. The resultant strains have a single copy of the plasmid fragment inserted stably at the lambda attachment site on the chromosome, with nearly the entire lambda genome deleted. The system for generating single-copy chromosomal versions of plasmid-encoded genes utilizes a bacteriophage lambda derivative which is named lambda InCh (for “into the chromosome”). The transfer requires three successive in vivo steps, but no in vitro cloning steps. Both homology-dependent recombination and site-specific recombination are involved. The steps required for this process are (i) recombination of the desired plasmid-encoded genes (promoter/reporter genes) onto lambda InCh; (ii) integration of the recombinant lambda InCh, carrying the newly incorporated genes, into the chromosome at the lambda attachment site; and (iii) deletion of most of the lambda genes, including one attachment site, from the chromosome of the lysogen. Details of the lambda strains and the process of integration are described in the Boyd paper.

Promoter/Reporter construction—The construction of the reporter and promoter constructs utilized both GFPuv (a UV excitable version of green fluorescent protein) and beta-glactosidase. These were cloned into a PUC-based vector downstream from the promoter sequence. All promoter sequences were obtained from E. coli K12 using standard PCR techniques. In all cases, the reporter was as expressed as a fusion of the first 4-7 amino acids of the native regulatory protein, which normally sits downstream from the homeostatic promoter, and the reporter protein (GFPuv or β-galactosidase). Strains were constructed using E. coli DH10B (Invitrogen). Identification of a successful single copy reporter strain was accomplished by monitoring antibiotic resistance, PCR of the unique sequences and activation of the promoter by control compounds.

Transformation of environmental libraries—Environmental libraries were transformed into the reporter strains using standard electroporation conditions (Bio-Rad). Cells were incubated overnight at 37, aliquots frozen, and samples analyzed for expression of the reporter genes at 24, 48 and 72 hours.

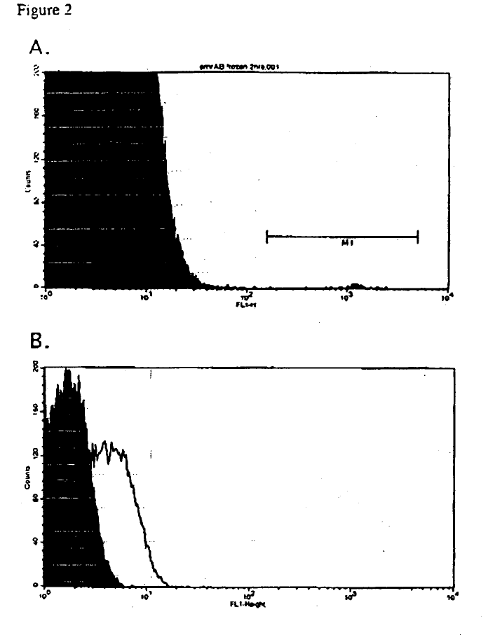

Flow cytometry—Isolation of clones expressing the reporter was accomplished on a FACS Vantage (Becton-Dickinson) using a 488 Argon laser and a 530 nm band pass filter. Positive were selected based on comparison to control bacteria transformed with vector alone (FIG. 2A). Collected clones were plated on selective LB-agar overnight and individual clones were re-analyzed for activity (FIG. 2B). Validation of positive clones was accomplished by isolating the environmental DNA plasmid clone and re-transforming it into naive reporter strains.

Results and Discussion

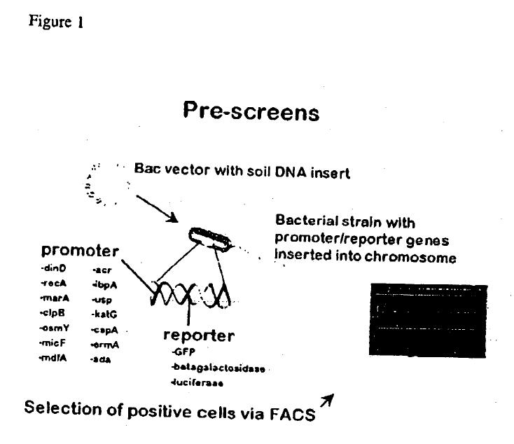

This approach utilizes the myriad of responses which exist in a host organism for dealing with stress brought on by many environmental and chemical stimuli. In particular, when a library bacterial clone produces a natural product (due to insertion and expression of environmentally derived DNA into the cell), the cell may respond by activating one or many stress response genes and/or proteins involved in multidrug transporters (MDT)( FIG. 1). This class of promoters have been defined herein as homeostatic promoters since they respond to alterations in the general homeostasis of the cell. A series of bacterial pre-screening strains have been created that contain gene fusions made up of a reporter gene (e.g., Green Fluorescent Protein or β-galactosidase) and a promoter for stress-response genes or MDT proteins. The gene fusions have been inserted into the chromosome of a bacterial cell, such as E. coli, so that only one copy exists per cell, and therefore are regulated in a normal fashion. The pre-screening strains are transformed with the environmental DNA libraries so that activation of the promoter following the expression of certain heterologous environmental genes/proteins will induce the reporter gene. The transformed library is screened using flow cytometry. As explained above, methods of the present invention can also be performed as a high-throughput method that allows the analysis of millions of clones in a relatively short period of time. The positive clones are sorted out and comprise a “high-quality” (HQ) library which is used for further detailed analysis, or transferred to another host strain. The HQ library is constantly being enlarged as libraries are being created and prescreened. Thus, methods of the present invention are dynamic as well as high-throughput.

Using this approach we transformed a 10,000 clone library which was constructed using soil DNA transformed into several promoter strains including katg, clpB, dinD and the novel variant emrAB promoter of the present invention (see below). The bacteria were grown and sorted on successive days and positive clones isolated from two of the strains (katg and emrAB). The relatively small library size (10,000) suggests that clones activating these promoters are rare (<1%) which is expected. As mentioned above, the method is designed to screen millions of bacterial clones relatively fast. Flow rates as high as 1000 cells/sec such that the analysis of a 1 million clone library requires approximately 15-30 minutes.

### EXAMPLE II

### Novel Promoter of the Present Invention

Materials and Methods

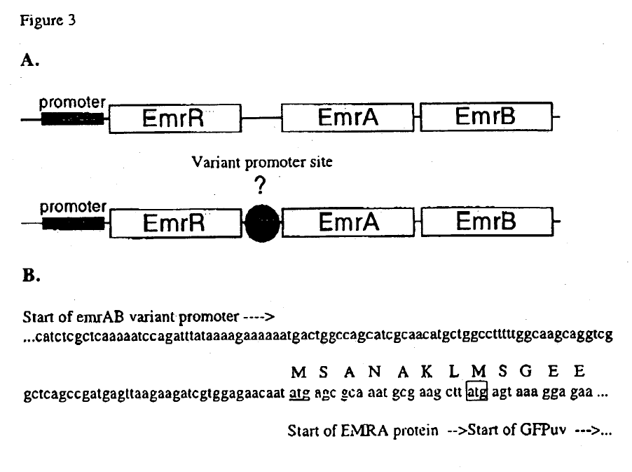

Promoter/Reporter construction—The variant emr AB promoter (v-emrAB) was selected from the intergenic region between the emrR and emrA genes in E. coli K12 (FIG. 3). This region is 126 bp in length and contains no readily identifiable promoter elements. The DNA fragment containing the promoter sequence was obtained from E. coli K12 using standard PCR techniques. The reporter was as expressed as a fusion of the first 7 amino acids of emrA and the reporter protein (GFPuv or β-galactosidase). Strains were constructed using E. coli DH10B (Invitrogen). Identification of a successful single copy reporter strain was accomplished by monitoring antibiotic resistance, PCR of the unique sequences and activation of the promoter by control compounds.

Results and Discussion

As described above, an environmental library composed of 10,000 individual clones was transformed into the v-emrAB strain and sorted on a FACS Vantage. A small population of positive clones was evident (FIG. 2A) and represented less than 0.1% of the total population. These clones were plated after sorting, grown overnight, and re-analyzed for GFPuv expression. FIG. 2B is an example of one positive clone compared to empty vector. The expression level of the GFPuv is lower. Moreover, several examples of environmental clones have been observed which activate the v-emrAB promoter at specific times after transformation. Although under no obligation to explain such results and not to be bound by such results, this temporal expression may be due to the normal control processes which affect this class of homeostatic promoters.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

### References

Lomovskaya et al., EmrR Is a Negative Regulator of the Escherichia coli Multidrug Resistance Pump EmrAB. J. Bacteriology, 177(9): 2328-2334 (1995).

Belkin, S. et al., Reporter gene bioassays in environmental analysis. Fresenius J Anal Chem 2000 March-April;366(6-7):769-79

Bianchi A A, Baneyx F., Stress responses as a tool To detect and characterize the mode of action of antibacterial agents. Appl Environ Microbiol 1999 November;65(11):5023-7.

Cha H J et al., Green fluorescent protein as a noninvasive stress probe in resting Escherichia coli cells. Appl Environ Microbiol 1999 February;65(2):409-14.

Ben-Israel, O. et al., Identification and quantification of toxic chemicals by use of Escherichia coli arrying lux genes fused to stress promoters. Appi Environ Microbiol 1998 November;64(11):4346-52.

Valdivia R H, Falkow S. Bacterial genetics by flow cytometry: rapid isolation of Salmonella typhimurium acid-inducible promoters by differential fluorescence induction. Mol Microbiol 1996 October;22(2):367-78.

Nishino K, Yamaguchi A., Analysis of a complete library of putative drug transporter genes in Escherichia coli. J Bacteriol 2001 October;183(20):5803-12.

Putman M, van Veen H W, Konings W N., Molecular properties of bacterial multidrug transporters.

Microbiol Mol Biol Rev 2000 December;64(4):672-93.

Lewis et al., PNAS 89:8938 (1992).

Lewis et al., J. Bact.177:2328 (1995).

Lewis et al., J. Bact. 181:5131

Lewis et al., Antimicrob. Agents Chemother. 44:2905 (2000).

Boyd, D. et al., Towards Single-Copy Gene Expression Systems Making Gene Cloning Physiologically Relevant: Lambda InCh, a Simple Escherichia coli Plasmid-Chromosome Shuttle System. J. Bacteriology 182:842-847 (2000).

What is claimed is:

1. A method for identifying a DNA molecule that encodes a natural product having bioactivity or a protein involved in the production of a natural product having bioactivity, comprising the steps of:
(a) providing a transformed bacterial cell comprising:
(i) a gene fusion incorporated into the transformed bacterial cell's genome so that only one copy of the gene fusion is present within the transformed bacterial cell, wherein the gene fusion comprises a DNA molecule that encodes for a reporter protein operatively associated with a homeostatic promoter which may be activated by the natural product, and
(ii) an exogenous DNA molecule that encodes for the natural product or the protein involved in the production of the natural product within the transformed bacterial cell;
(b) incubating the transformed bacterial cell under conditions that permit the production of the natural product; and
(c) detecting the presence of the reporter protein within the transformed bacterial cell,
wherein the detection of the reporter protein within the transformed bacterial cell indicates that the exogenous DNA molecule encodes a natural product having bioactivity or a protein involved in the production of a natural product having bioactivity.

2. The method of claim 1, wherein the transformed bacterial cell comprises an E. coli cell, a Bacillus cell, a Staphylococcus cell, a Streptomyces cell, a Myxobacteria cell, or a Pseudomonas cell.

3. The method of claim 1, wherein the reporter protein comprises green fluorescent protein, β-galactosidase, or luciferase.

4. The method of claim 1, wherein the homeostatic promoter is a stress promoter or a multidrug transporter promoter.

5. The method of claim 4, wherein the stress promoter is selected from the group consisting of: katG, micF, osmY, uspA, ibpA, rpoH, recA, clpB, gyrA, ada and dinD.

6. The method of claim 4, wherein the multidrug transporter promoter is selected from the group consisting of: EmrD, MdfA, EmrAB, EmrE, AcrAB, RAB, acrD, acrEF, and emrR.

7. The method of claim 1, wherein the exogenous DNA molecule is contained within a bacterial artificial chromosome with which the transformed bacterial cell is transformed.

8. The method of claim 7, wherein the exogenous DNA molecule is an environmentally-derived DNA molecule.

9. A method for identifying a DNA molecule that encodes a natural product having bioactivity or a protein involved in the production of a natural product having bioactivity, comprising the steps of:
(a) providing a transformed E. coli cell comprising:
(i) a gene fusion incorporated into the transformed E. coli cell's genome so that only one copy of the gene fusion is present within the transformed E. coli cell, wherein the gene fusion comprises a DNA molecule that encodes for a reporter protein operatively associated with a homeostatic promoter which may be activated by the natural product, and
(ii) a bacterial artificial chromosome comprising an exogenous DNA molecule that encodes for the natural product or the protein involved in the production of the natural product within the transformed E. coli cell;
(b) incubating the transformed E. coli cell under conditions that permit the production of the natural product; and
(c) detecting the presence of the reporter protein within the transformed E. coli cell,
wherein the detection of the reporter protein within the transformed E. coli cell indicates the exogenous DNA molecule encodes a natural product having bioactivity or a protein involved in the production of a natural product having bioactivity.

10. The method of claim 9, wherein the exogenous DNA molecule is an environmentally-derived DNA molecule.

11. The method of claim 10, wherein the homeostatic promoter is a stress promoter or a multidrug transporter promoter.

12. The method of claim 11, wherein the stress promoter is selected from the group consisting of: katG, micF, osmY, uspA, ibpA, rpoH, recA, clpB, gyrA, ada and dinD.

13. The method of claim 11, wherein the multidrug transporter promoter is selected from the group consisting of: EmrD, MdfA, EmrAB, EmrE, AcrAB, RAB, acrD, acrEF, and emrR.

14. The method of claim 11, wherein the reporter protein comprises green fluorescent protein, β-galactosidase, or luciferase.

15. The method of claim 14, wherein the reporting protein is green fluorescent protein and the detecting step is performed using flow cytometry.

16. A method for identifying an environmentally-derived DNA molecule that encodes a natural product having bioactivity or a protein involved in the production of a natural product having bioactivity, comprising the steps of:
(a) providing a transformed E. coli cell comprising:
(i) a gene fusion incorporated into the transformed E. coli cell's genome so that only one copy of the gene fusion is present within the transformed E. coli cell, wherein the gene fusion comprises a DNA molecule that encodes green fluorescent protein, operatively associated with a homeostatic promoter which may be activated by the natural product, and
(ii) a bacterial artificial chromosome comprising the environmentally-derived DNA molecule that encodes for the natural product or a protein involved in the production of the natural product within the transformed E. coli cell,
(b) incubating the transformed E. coli cell under conditions that permit the production of the natural product; and
(c) detecting with cytometry the green fluorescent protein within the transformed E. coli cell,
wherein the detection of the green fluorescent protein within the transformed E. coli cell indicates the environmentally-derived DNA molecule encodes a natural product having bioactivity or a protein involved in the production of a natural product having bioactivity.

17. A method for separating environmentally-derived DNA molecules that encode natural products having bioactivity or proteins involved in the production of natural products having bioactivity from environmentally-derived DNA molecules that do not encode natural products having bioactivity or proteins involved in the production of natural products having bioactivity, comprising the steps of:
(a) providing transformed bacterial cells wherein each transformed bacterial cell comprises a gene fusion incorporated into the transformed bacterial cell's genome so that only one copy of the gene fusion is present within each transformed bacterial cell, the gene fusion comprising a DNA molecule that encodes for a reporter protein operatively associated with a homeostatic promoter which may be activated by a natural product;
(b) transforming each transformed bacterial cell with a bacterial artificial chromosome comprising an environmentally-derived DNA molecule that encodes for the natural product or a protein involved in the production of the natural product so that each bacterial artificial chromosome comprises only one environmentally-derived DNA molecule;
(c) incubating the cells under conditions that permit the production of the natural products; and
(d) separating the transformed bacterial cells in which the reporter protein is detected from the transformed bacterial cells in which the reporter protein is not detected,
such that the separated transformed bacterial cells in which the reporter protein is detected contain the environmentally-derived DNA molecules that encode natural products having bioactivity, or proteins involved in the production of natural products having bioactivity.

18. The method of claim 17, wherein the transformed bacterial cells are E. coli cells.

19. The method of claim 17, wherein the homeostatic promoter comprises a stress promoter or a multidrug transporter promoter.

20. The method of claim 19, wherein the stress promoter is selected from the group consisting of: katG, micF, osmY, uspA, ibpA, rpoH, recA, clpB, gyrA, ada and dinD.

21. The method of claim 19, wherein the multidrug transporter promoter is selected from the group consisting of: EmrD, MdfA, EmrAB, EmrE, AcrAB, RAB, acrD, acrEF, and emrR.

22. The method of claim 17, wherein the reporter protein comprises green fluorescent protein, β-galactosidase, or luciferase.

23. The method of claim 22, wherein the reporter protein is green florescent protein, and the separating step is performed with flow cytometry.

24. The method of claim 23, wherein the method is a high throughput method.

25. A method for separating environmentally-derived DNA molecules that encode natural products having bioactivity or proteins involved in the production of natural products having bioactivity from environmentally-derived DNA molecules that do not encode natural products having bioactivity or proteins involved in the production of natural products having bioactivity, comprising the steps of:
(a) providing transformed E. coli cells comprising a gene fusion incorporated into the genome of each transformed E. coli cell so that only one copy of the gene fusion is present within each transformed E. coli cell, the gene fusion comprising a DNA molecule that encodes for green fluorescent protein operatively associated with a homeostatic promoter which may be activated by a natural product;
(b) transforming each transformed E. coli cell with a bacterial artificial chromosome comprising an environmentally-derived DNA molecule that encodes the natural product or a protein involved in the production of the natural product, so that each bacterial artificial chromosome comprises only one environmentally-derived DNA molecule;
(c) incubating the transformed E. coli cells under conditions that permit the production of the natural products in the transformed bacterial cells; and
(d) separating the transformed E. coli cells in which the green fluorescent protein is detected from the transformed E. coli cells in which the green fluorescent protein is not detected,
such that the separated transformed E. coli cells in which the green fluorescent protein is detected contain the environmentally-derived DNA molecules that encode natural products having bioactivity, or proteins involved in the production of natural products having bioactivity.

26. A promoter for controlling the expression of a DNA molecule that encodes an amino acid sequence, wherein contact between said promoter and an organic solvent activates said promoter, and said promoter consists essentially of a DNA sequence of

| | |
|---|---|
| CGCGTCATCTCGCTCAAAAATCCAGATTTATAAAAGAAAAAATGACTGGCCAGCA | (SEQ ID NO:20) |
| TCGCAACATGCTGGCCTTTTTGGCAAGCAGGTCGGCTCAGCCGATGAGTTAAGAA | |
| GATCGTGGAGAACAAT. | |

27. A method for identifying an environmentally derived DNA molecule that encodes a natural product that inhibits a homeostatic response or encodes a protein involved in the production of a natural product that inhibits a homeostatic response, comprising the steps of:
(a) providing a transformed bacterial cell comprising a gene fusion incorporated into the genome of the transformed bacterial cell so that only one copy of the gene fusion is present within the transformed bacterial cell, the gene fusion comprising a DNA molecule that encodes a reporter protein operatively associated with a homeostatic promoter;
(b) transforming the transformed bacterial cell with a bacterial artificial chromosome comprising an environmentally-derived DNA molecule that encodes a natural product or a protein involved in the production of the natural product;
(c) incubating the cell incubated under conditions that permit the production of the natural product;
(d) contacting the cell with a known inducer of the homeostatic promoter in order to activate the cell; and
(e) examining the for the presence of the reporter protein,
wherein lack of detection of the protein in the cell indicates that the environmentally derived DNA molecule a natural product or protein involved in the production of a natural product that inhibits the normal alterations in homeostasis.
